(19) United States
(12) Patent Application Publication
(10) Pub. No.: US 2023/0073758 A1
(43) Pub. Date: Mar. 9, 2023

(54) SHAVER ARRANGEMENT FOR A SURGICAL INSTRUMENT

(71) Applicant: GYRUS MEDICAL LIMITED, Cardiff (GB)

(72) Inventor: James Alan DICKSON, Cardiff (GB)

(73) Assignee: GYRUS MEDICAL LIMITED, Cardiff (GB)

(21) Appl. No.: 17/892,786

(22) Filed: Aug. 22, 2022

Related U.S. Application Data
(60) Provisional application No. 63/241,767, filed on Sep. 8, 2021.

(51) Int. Cl. A61B 17/32; A61B 18/14

(52) U.S. Cl. CPC A61B 17/32002; A61B 18/148; A61B 2018/00607

(57) ABSTRACT

A shaver arrangement for a surgical instrument includes an outer member and cutting blade at a distal end; the cutting blade being serrated to form teeth forming one or more tissue engaging cutting points being arranged to snag and/or cut tissue and being located inwardly from the outer member such that there are no tissue engaging cutting points capable in use of snagging and/or cutting tissue located on or outwards of an outer surface of the outer member. The shaver arrangement reduces the likelihood of collateral damage to surrounding non-target tissue. The shaver arrangement does not have tissue engagement cutting points on the outer profile of the cutting blade of the shaver arrangement, thereby reducing the likelihood of collateral damage when manoeuvring the surgical instrument as tissue is less likely to be inadvertently caught by the tissue cutting points.

### TECHNICAL FIELD

Embodiments of the present disclosure described herein relate to surgical devices, and in particular to a shaver arrangement for a surgical instrument and an end effector for an electrosurgical instrument.

### BACKGROUND TO THE INVENTION AND PRIOR ART

Many shavers, for example, arthroscopic shavers, have blades with teeth on either the inner blade, outer blade, or both. Typically, the more teeth, and the sharper the teeth, the more aggressive the blade is assumed to be during clinical use. Very aggressive blades must also be carefully positioned within the clinical joint space to avoid any collateral tissue damage to healthy tissue. In traditional arthroscopic shavers, there is generally only one shave window with teeth, and therefore the opposite side of the end effector is smooth. This allows surgeons to slide over healthy tissue with the smooth side, while keeping the sharp teeth away from healthy tissue when accessing tight spaces.

In a dual sided RF shaver construction, both sides of the tip have a function, and therefore neither side is perfectly smooth. Therefore, a fundamental conflict exists for an ultra-aggressive blade. During shaver use, the blade should be as aggressive as a typical toothed outer blade, but when the user is operating the RF side of the active tip, the teeth should not cause any collateral tissue damage to healthy tissue when the blade window is closed and the shaver is stationary.

Typical inner and outer teeth profiles for ultra-aggressive shaver shown in FIG. 1. In the generic shaver design shown in FIG. 1, there is a sharp tooth edge on the inside of the outer blade 102a, 102b which actively shear tissue against the spinning inner blade, but there is also a sharp outer surface tooth edge 101a, 101b.

### SUMMARY OF THE DISCLOSURE

The present disclosure addresses the above problem, by providing a shaver arrangement for a surgical instrument wherein the outer profile of the cutting blade located on the outer tubular member is smooth, such that it greatly reduces the likelihood of the cutting blade snagging or interacting with non-target tissue (i.e. causing collateral damage). In other words, with reference to the prior art FIG. 1, the present disclosure removes the sharper outer surface tooth edges 101a, 101b by smoothing out the outer profile of the cutting blade. This smoother outer profile may be in the form of a chamfered edge and/or radius edge. The radius edge may be a constant radius. The same function as a chamfered edge could be obtained by choosing a large radius dimension, which approaches a flat chamfer in reality.

Embodiments of the present disclosure provide a shaver arrangement for a surgical instrument which reduces the likelihood of collateral damage to surrounding non-target tissue. The arrangement does not have tissue engagement cutting points on the outer profile of the cutting blade of the arrangement, thereby reducing the likelihood of collateral damage when manoeuvring the surgical instrument as tissue is less likely to be inadvertently caught by the tissue cutting points.

In view of the above, from a first aspect, the present disclosure relates to a shaver arrangement for a surgical instrument, the shaver arrangement comprising an outer member and a cutting blade at a distal end thereof. The cutting blade being serrated to form a plurality of teeth, each of the teeth forming one or more tissue engaging cutting points. The one or more tissue engaging cutting points being arranged in use to snag and/or cut tissue. The tissue engaging cutting points being located inwardly from the outer member such that there are no tissue engaging cutting points capable in use of snagging and/or cutting tissue located on or outwards of an outer surface of the outer member. Several advantages are obtained from embodiments according to the above described aspect. For example, the tissue engaging cutting points are arranged such that they interact only with tissue to be treated, not healthy surrounding tissue.

In some embodiments, each of the plurality of teeth has a tooth peak, and the tooth peak forms one of the one or more tissue engaging cutting points.

In some embodiments, there are no tissue engaging cutting points located outwards of an inner surface of the outer member.

This is advantageous as by only having tissue engaging cutting points located outwards of an inner surface of the outer member, the chance of the tissue engaging cutting points catching healthy surrounding tissue and causing collateral damage is greatly reduced.

In some embodiments, the cutting blade has a chamfered edge and/or radius edge. This results in an arrangement where the tissue engaging cutting points are located inwards from the outer member such that there are no tissue engaging cutting points capable in use of snagging and/or cutting tissue located on or outwards of an outer surface of the outer member. The chamfered edge and/or radius edge may connect an outer surface of the cutting blade to an inner surface of the cutting blade. The chamfered edge and/or radius edge may be angled such that the inner surface of the cutting blade extends further than the outer surface of the cutting blade. The one or more tissue engaging cutting points may be located at the inner surface of the cutting blade. The chamfered edge and/or radius edge may be angled at an angle between 10 and 60 degrees with respect to an axial plane to a longitudinal plane of the shaver arrangement. The angle may be between 30 and 50 degrees.

In some embodiments, the shaver arrangement is a rotary shaver arrangement, the outer member is an outer tubular member with a cutting window at the distal end thereof, the cutting window has at least one sharpened edge to form the cutting blade, and the tissue engaging cutting points are located radially inwards from the outer tubular member such that there are no tissue engaging cutting points capable in use of snagging and/or cutting tissue located on or outwards of an outer circumference of the outer tubular member.

In some embodiments, the outer tubular member has a central passageway and the cutting window is a first cutting window, and the arrangement further comprises an inner tubular member rotatably mounted in the central passageway of the outer tubular member and providing a central suction lumen, the inner tubular member having a second cutting window at the distal end thereof such that the first and second cutting windows align when the inner tubular member is rotated to a first position. The arrangement may be such that, when in use, rotation of the inner tubular member within the outer tubular member causes a tissue cutting action of the cutting blade interacting with the second cutting window.

In some embodiments, the shaver arrangement of any of the above embodiments is incorporated into an end effector for an electrosurgical instrument, the end effector additionally comprising an RF arrangement including an active electrode.

From a second aspect, the present disclosure relates to an end effector for an electrosurgical instrument, the end effector comprising an RF arrangement including an active electrode; and a shaver arrangement comprising an outer member and a cutting blade at a distal end thereof. The cutting blade is arranged to have one or more tissue engaging cutting points, the one or more tissue engaging cutting points being arranged in use to snag and/or cut tissue, the tissue engaging cutting points being located inwardly from the outer member such that there are no tissue engaging cutting points capable in use of snagging and/or cutting tissue located on or outwards of an outer surface of the outer member.

The above embodiments relating to the first aspect may equally apply to the second aspect.

In some embodiments, the shaver arrangement is such that, when the RF arrangement is in use, there is a reduced likelihood of the shaver arrangement engaging with tissue. This is advantageous as, for example, for dual sided RF shavers, the RF arrangement and the shaver arrangement may be located at different circumferential locations around the end effector's tubular members (e.g. on opposite sides), and so when the RF arrangement is being used to treat tissue and the shaver arrangement is not in use, the movement of the end effector does not cause tissue engagement points of the shaver arrangement to catch on non-target tissue and cause collateral damage.

In some embodiments, the cutting blade is serrated to form a plurality of teeth, each of the plurality of teeth forming one of the one or more tissue engaging cutting points. Each of the plurality of teeth may have a tooth peak, and the tooth peak may form one of the one or more tissue engaging cutting points.

From a third aspect, the present disclosure relates to an electrosurgical instrument comprising: an end effector according to the last embodiment of the first aspect described above, or any embodiment of the second aspect described above; and an operative shaft having RF electrical connections operably connected to the active electrode, and drive componentry operably connected to the shaver arrangement to drive the shaver arrangement to operate in use.

From a fourth aspect, the present disclosure relates to an electrosurgical system, comprising: an RF electrosurgical generator; and an electrosurgical instrument according to the third aspect, the arrangement being such that in use the RF electrosurgical generator supplies an RF coagulation or ablation signal via the RF electrical connections to the active electrode.

### BRIEF DESCRIPTION OF THE DRAWINGS

Embodiments of the invention will now be further described by way of example only and with reference to the accompanying drawings, wherein:

FIG. 1 illustrates prior art inner and outer teeth profiles for an ultra-aggressive shaver;

FIG. 2 is a schematic diagram of an electrosurgical system including an electrosurgical instrument;

FIG. 3 is an end view of an electrosurgical instrument illustrating embodiments of the present invention;

FIG. 4 is a side view of an electrosurgical instrument illustrating embodiments of the present invention;

FIG. 5 is a top view of a rotary shaver arrangement illustrating embodiments of the present invention;

FIG. 6 is a perspective view of a rotary shaver arrangement illustrating embodiments of the present disclosure where the blade window is closed during use of the RF arrangement, thereby creating a smooth outer surface on the rotary shaver arrangement side;

FIG. 7 is a perspective view of a rotary shaver arrangement illustrating embodiments of the present invention; and

FIGS. 8a and 8b are a comparison between an end view of an electrosurgical instrument illustrating prior art arrangements (FIG. 8a) and an end view of an electrosurgical instrument illustrating embodiments of the present disclosure (FIG. 8b).

### DESCRIPTION OF THE EMBODIMENTS

Overview

Embodiments of the present disclosure modify and improve upon prior art designs of shaver arrangements by smoothing the outer profile of the sharpened edge used to cut tissue. This sharpened edge is located within/on the outer member of the shaver arrangement, and in use, movement of an inner member with respect to the outer member causes a tissue cutting action of the inner surface of the cutting blade interacting with the inner member. There is no need for the outer surface of the sharpened edge to be a tissue engaging point, and as described above, by having tissue engaging points on the outer surface of the sharpened edge, there is a significant possibility of collateral damage to nearby tissue.

Embodiments of the present disclosure provide a shaver arrangement wherein the sharpened edge/cutting blade has a smooth outer profile to prevent tissue catching on the outer surfaces of the blade. In other words, the outer profile of the cutting blade does not have any tissue engaging cutting points. Only the inner profile of the cutting blade has tissue engaging cutting points.

The smooth outer profile may be in the form of a chamfered and/or radius edge. In the case of a chamfered edge, the chamfer is in the axial plane to the shaver rotational plane. The inside edge shearing tooth (the tissue engaging point on the inner surface of the outer tubular member) is still present, and the inner cutting blade geometry remains unchanged as this is hidden during RF activation. However, the outer tooth that presents more of a collateral damage risk during opposite sided RF use is removed from the geometry using a chamfer/blend to the outer surface of the blade. The resulting geometry with a closed shave window is a smoother profile, less likely to damage surrounding tissue during opposite sided RF tip use.

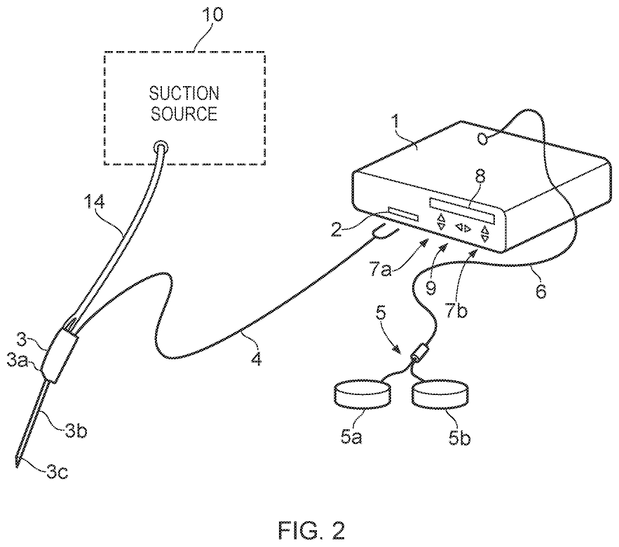

Various aspects and details of these principal components will be described below by way of example with reference to FIGS. 2 to 8b.

The Electrosurgical System

Referring to the drawings, FIG. 2 shows an electrosurgical system including an electrosurgical generator 1 having an output socket 2 providing an RF output, via a connection cord 4, for an electrosurgical instrument 3. The instrument 3 has a suction tube 14 which is connected to suction pump 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

The Electrosurgical Instrument

The instrument 3 includes a proximal handle portion 3a, a shaft 3b extending in a distal direction away from the proximal handle portion, and a distal end effector assembly 3c at the distal end of the shaft 3b. A power connection cord 4 connects the instrument to the RF generator 1. The instrument may further be provided with activation buttons (not shown), to allow the surgeon operator to activate either the mechanical cutting function of the end effector, or the electrosurgical functions of the end effector, which typically comprise coagulation or ablation.

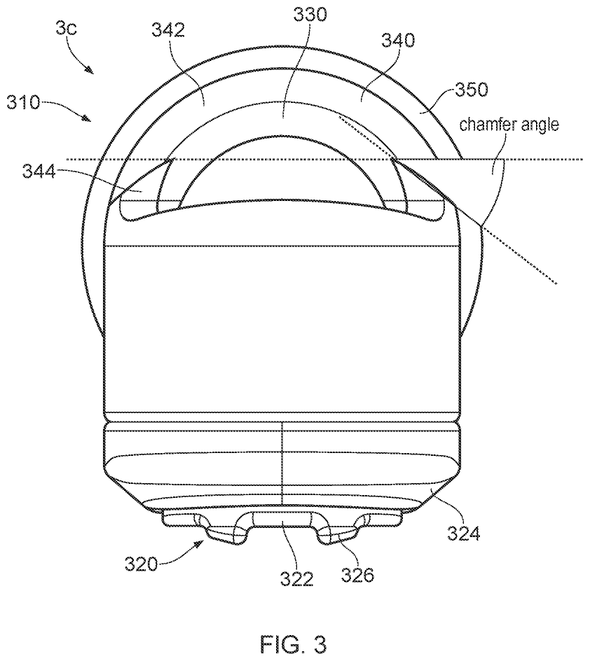

FIGS. 3-7 show an example of a distal end effector assembly 3c in more detail. The example shown is a dual-sided RF rotary shaver, however, embodiments of the invention may be applied to other shaver arrangements including shavers without RF capabilities, reciprocating shavers and rotary shavers. The distal end effector 3c has two sides to it, the shaver side 310 and the RF side 320. The shaver side 310 is on the opposite side of the end effector to the RF side 320. The shaft 3b comprises an inner tubular member 330 and an outer tubular member 340. The outer tubular member 340 may be concentrically surrounded by an insulative tubular member 350.

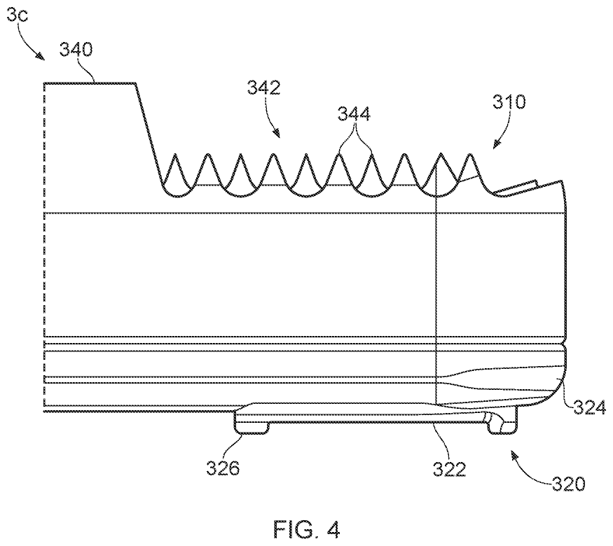

FIG. 3 shows an end view of the distal end effector assembly 3c. In FIG. 3, the blade window is closed. The inner tubular member 330 is co-axially disposed within an outer tubular member 340. The outer tubular member 340 has a larger diameter than the inner tubular member 330. The inner tubular member 330 has a proximal end and a distal end, with cutting window 332 (not visible in FIG. 3) disposed at a side of its distal end. The outer tubular member 340 also has a proximal end and a distal end, with cutting window 342 (best shown in FIG. 4) disposed at a side of its distal end. The edges of the cutting window 342 of the outer tubular member 340 comprise at least one sharpened edge to form a cutting blade. The at least one sharpened edge may be serrated such that it comprises teeth 344, as shown in the example given in the Figures.

The teeth 344 have a slanted outer profile. The slanted outer profile of the teeth 344 is created by adding a chamfered/bevelled/radius edge to the outer tooth profile only. The chamfered edge is in the axial plane to the shaver rotational plane. The resulting geometry when the blade/cutting window is closed is a smoother profile, resulting in a lower likelihood of damage to surrounding tissue during use of the RF side 310. However, it will be appreciated that having a serrated edge with a plurality of teeth is not a requirement to implement certain embodiments of the present invention. The slanted outer profile concept may be similarly applied to non-serrated sharpened edges. For example, the sharpened edge may look like FIG. 3 from the end view, but then be a plain non-serrated edge without individual teeth, which would be visible from a side view or a perspective view. The sharpened edge would still have the slanted outer profile as described herein, i.e. a chamfered/bevelled/radius edge.

The inner tubular member 330 is rotatably disposed inside of the outer tubular member 340 such that the surgical instrument 3 cuts tissue by rotating the inner tubular member 330 within the outer tubular member 340 while a vacuum is applied through the lumen of the inner tubular member 330 to draw the tissue into the cutting windows and sever the tissue by rotation of the inner tubular member.

The RF side 320 of the electrosurgical instrument 3 comprises an electrode assembly comprising an active electrode for tissue treatment (“active tip”) 322 received in a ceramic insulator 324. The active tip 322 is provided with projections 326 to concentrate the electric field at those locations. The projections 326 also serve to create a small separation between the planar surface of the active electrode 322 and the tissue to be treated. This allows conductive fluid to circulate over the planar surface, and avoids overheating of the electrode or the tissue. The active tip 322 of the instrument may be provided with a suction aperture (not shown), which is the opening to a lumen within an inner tubular member 330.

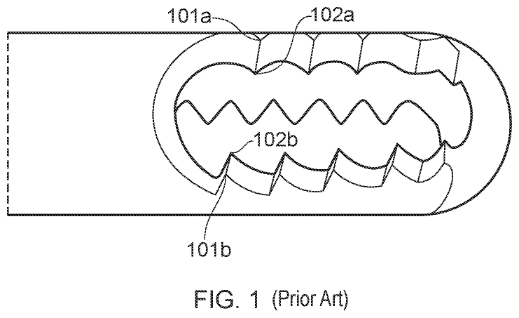

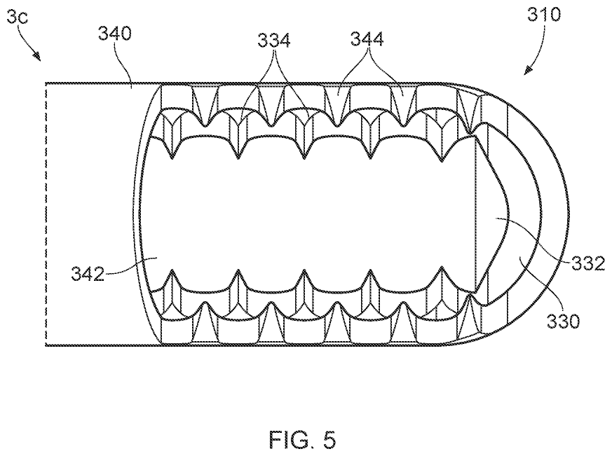

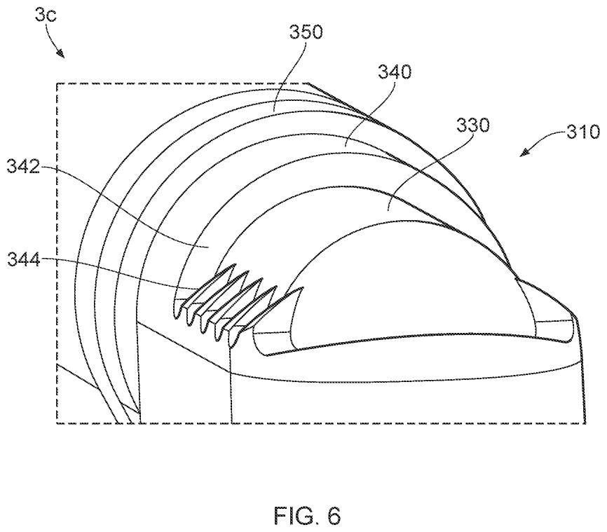

In more detail, when the RF side 320 is to be used as a suction tool by applying a vacuum through the lumen within the inner tubular member 330, the inner tubular member 330 (which acts as a cutting blade) is stopped from rotating and the cutting windows of the inner and outer tubular members are misaligned with each other, i.e. closing the cutting windows, (as is the case in FIGS. 5 and 6) so that the vacuum is applied through the suction path connecting the suction aperture to the suction pump 10 via the lumen to transport fluids to and from the active tip 322. It is during this use that a key advantage of the invention becomes apparent. In the prior art, outer tissue engagement points (see 101a, 101b in FIG. 1), would be likely to snag and/or cut non-target tissue when the instrument is manoeuvred when using the RF functionality even when the cutting window is closed. In contrast, embodiments of the present disclosure do not have these outer tissue engagement points due to their smooth outer profile. As such, there is a greatly reduced likelihood of snagging and/or cutting non-target tissue when the instrument is manoeuvred using the RF functionality.

In contrast, when the shaver side 310 is in use for a cutting operation, suction may flow via through the cutting windows to the lumen.

The inner and outer tubular members 330 and 340 may be made from stainless steel. Stainless steel is a good option as it is easy to bond to the steel cuttings tips of inner and outer tubular members, which act as the RF return, and due to the blade properties of hardened steel.

FIG. 4 shows a side view of the distal end effector assembly 3c. In FIG. 4, the blade window is open. In FIG. 4, the cutting window 342 of the outer tubular member 340 is better shown. Outer teeth 344 line the edge of the outer cutting window 342. The inner tubular member 330 is not visible in FIG. 4 as the blade window is open such that the inner tubular member's cutting window 332 lines up with the outer tubular member's cutting window 342. The inner tubular member 330 is therefore hidden by the outer tubular member 340 in this view. The tooth side profile as shown in FIG. 4 is unchanged with respect to the prior art arrangement illustrated in FIG. 1.

FIG. 5 shows a top view of the shaver side 310 of the distal end effector assembly 3c. In FIG. 5, the blade window is open. In FIG. 5, the cutting windows of the inner and outer tubular members (332 and 342 respectively) are aligned such that the blade window is open. In some embodiments, there may be inner teeth 334 along the edge of the inner cutting window 332. In other embodiments, there may be no teeth along the edge of the inner cutting window 332, leaving a smooth surface.

FIG. 6 shows a perspective view of the shaver side 310 of the distal end effector assembly 3c. In FIG. 6, the blade window is closed (as would be the case during RF treatment). When the blade window is closed, a smooth outer surface is created by the inner tubular member 330. The inner cutting window 332 is not visible in FIG. 6, as it is misaligned with the outer cutting window 342, i.e. the inner cutting window 332 is rotated such that it is facing the opposite direction to the outer cutting window 342.

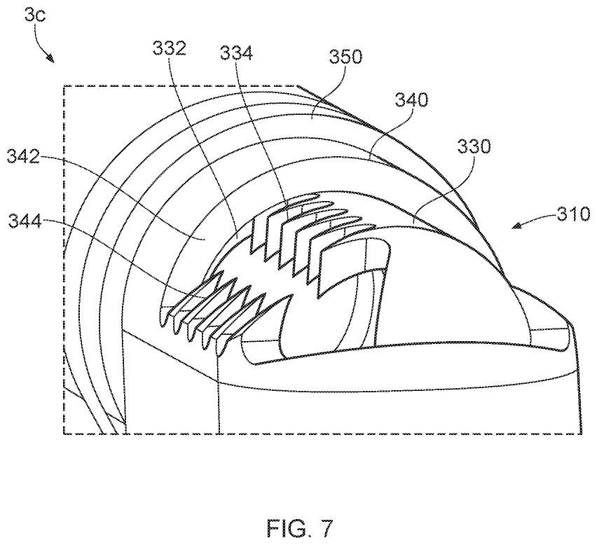

FIG. 7 shows a perspective view of the shaver side 310 of the distal end effector assembly 3c. In FIG. 7, the blade window is closing (as would be the case during shaver rotation). In FIG. 7, the inner tubular member 330 is in the process of rotating around the axis of the instrument (i.e. the central axis of the inner/outer tubular member).

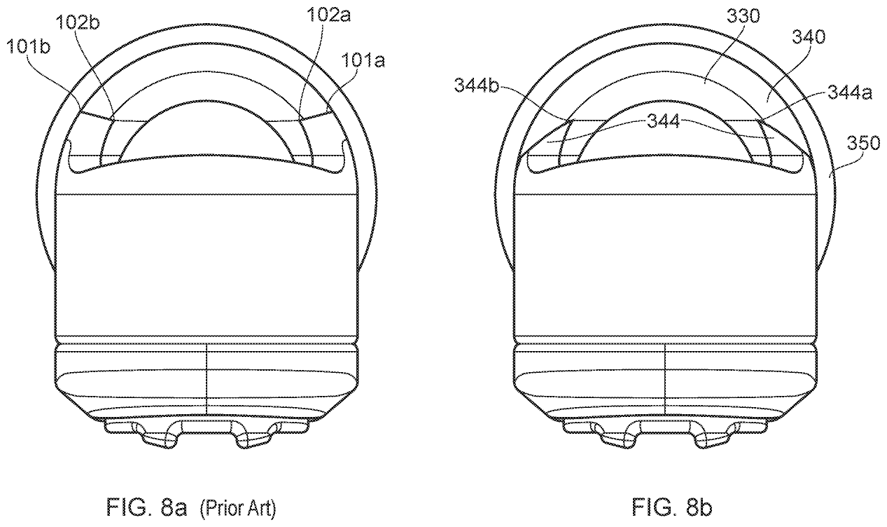

FIGS. 8a and 8b are a comparison between the outer teeth known in the prior art (FIG. 8a) and the outer teeth according to embodiments of the present disclosure (FIG. 8b). In both FIGS. 8a and 8b there are sharp tooth points on the inside of the outer tubular member 102b, 102a, 344b, 344a. These points actively shear tissue against the spinning inner tubular member 330, as described above. In the prior art arrangement (FIG. 8a), there are also sharp tooth points on the outside of the outer blade 101b, 101a, which, in use, may cause collateral damage to healthy tissue. This may occur when the RF side of the instrument is in use. When the RF side is in use, the cutting window is closed and the inner tubular member 330 is stationary. As the instrument is manoeuvred in use, healthy tissue may accidentally be caught on the outer sharp tooth points 101b, 101a, thereby causing collateral damage to the healthy tissue.

Embodiments of the present disclosure (FIG. 8b) overcome this problem found in arrangements of the prior art (e.g. FIG. 8a) by having teeth 344 with a slanted outer profile, such that there is only one tissue engagement point 344b on each tooth 344. The one point 344b is a sharp tooth point on the inside of the outer tubular member 340, and so the outer sharp tooth points of the prior art arrangements 101b, 101a are eliminated. This greatly reduces the risk of collateral damage to healthy tissue during use of the opposite RF side of the instrument.

It will be apparent to the skilled person that although one advantageous use of the invention is in a dual-sided RF shaver, the invention may also be applied to instruments without RF capabilities, for example, reciprocating shavers, rotary shavers and/or arthroscopic shavers.

From one aspect, the shaver arrangement does not have to have RF capabilities and is formed of a cutting blade being serrated to form a plurality of teeth. Each of the teeth form one or more tissue engaging cutting points. The one or more tissue engaging cutting points are arranged in use to snag and/or cut tissue. However, the tissue engaging cutting points are located inwardly from the outer member which the cutting blade is located on such that there are no tissue engaging cutting points capable in use of snagging and/or cutting tissue located on or outwards of an outer surface of the outer member.

From a second aspect, there is an end effector which has an RF arrangement and a shaver arrangement. The shaver arrangement does not need to have a cutting blade being serrated to form a plurality of teeth. However, the cutting blade is arranged to have one or more tissue engaging cutting points, the one or more tissue engaging cutting points being arranged in use to snag and/or cut tissue, the tissue engaging cutting points being located inwardly from the outer member such that there are no tissue engaging cutting points capable in use of snagging and/or cutting tissue located on or outwards of an outer surface of the outer member.

Therefore, there are predominantly two aspects of the invention. Firstly, a shaver arrangement with teeth, the teeth arranged so that they do not snag and/or cut non-target tissue. Secondly, an end effector which has RF capabilities and a shaver arrangement with a cutting blade arranged such that it does not snag and/or cut non-target tissue.

Various modifications whether by way of addition, deletion, or substitution of features may be made to above described embodiment to provide further embodiments, any and all of which are intended to be encompassed by the appended claims.

1. A shaver arrangement for a surgical instrument, the shaver arrangement comprising:
an outer member and a cutting blade at a distal end thereof;
the cutting blade being serrated to form a plurality of teeth, each of the teeth forming one or more tissue engaging cutting points, the one or more tissue engaging cutting points being arranged in use to snag and/or cut tissue, the tissue engaging cutting points being located inwardly from the outer member such that there are no tissue engaging cutting points capable in use of snagging and/or cutting tissue located on or outwards of an outer surface of the outer member.

2. A shaver arrangement according to claim 1, wherein each of the plurality of teeth has a tooth peak, and the tooth peak forms one of the one or more tissue engaging cutting points.

3. A shaver arrangement according to claim 1, wherein there are no tissue engaging cutting points located outwards of an inner surface of the outer member.

4. A shaver arrangement according to claim 1, wherein the cutting blade has a chamfered edge and/or a radius edge.

5. A shaver arrangement according to claim 4, wherein the chamfered edge and/or radius edge connects an outer surface of the cutting blade to an inner surface of the cutting blade.

6. A shaver arrangement according to claim 5, wherein the chamfered edge and/or radius edge is angled such that the inner surface of the cutting blade extends further than the outer surface of the cutting blade.

7. A shaver arrangement according to claim 5, wherein the one or more tissue engaging cutting points are located at the inner surface of the cutting blade.

8. A shaver arrangement according to claim 4, wherein the chamfered edge and/or radius edge is angled at an angle between 10 and 60 degrees with respect to an axial plane to a longitudinal plane of the shaver arrangement.

9. A shaver arrangement according to claim 1, wherein:
the shaver arrangement is a rotary shaver arrangement;
the outer member is an outer tubular member with a cutting window at the distal end thereof;
the cutting window has at least one sharpened edge to form the cutting blade; and
the tissue engaging cutting points are located radially inwards from the outer tubular member such that there are no tissue engaging cutting points capable in use of snagging and/or cutting tissue located on or outwards of an outer circumference of the outer tubular member.

10. A shaver arrangement according to claim 9, wherein the outer tubular member has a central passageway and the cutting window is a first cutting window, and the arrangement further comprises an inner tubular member rotatably mounted in the central passageway of the outer tubular member and providing a central suction lumen, the inner tubular member having a second cutting window at the distal end thereof such that the first and second cutting windows align when the inner tubular member is rotated to a first position.

11. An end effector for an electrosurgical instrument, the end effector comprising:
a shaver arrangement according to claim 1; and
a radio frequency (RF) arrangement including an active electrode

12. An end effector for an electrosurgical instrument, the end effector comprising:
a radio frequency (RF) arrangement including an active electrode; and
a shaver arrangement comprising an outer member and a cutting blade at a distal end thereof;
the cutting blade being arranged to have one or more tissue engaging cutting points, the one or more tissue engaging cutting points being arranged in use to snag and/or cut tissue, the tissue engaging cutting points being located inwardly from the outer member such that there are no tissue engaging cutting points capable in use of snagging and/or cutting tissue located on or outwards of an outer surface of the outer member.

13. An end effector according to claim 11, wherein the shaver arrangement is such that, when the RF arrangement is in use, there is a reduced likelihood of the shaver arrangement engaging with tissue.

14. An end effector according to claim 12, wherein the cutting blade is serrated to form a plurality of teeth, each of the plurality of teeth forming one of the one or more tissue engaging cutting points.

15. An end effector according to claim 12 wherein each of the plurality of teeth has a tooth peak, and the tooth peak forms one of the one or more tissue engaging cutting points.

16. An end effector according to claim 12, wherein there are no tissue engaging cutting points located outwards of an inner surface of the outer member.

17. An end effector according to claim 12, wherein the cutting blade has a chamfered edge and/or a radius edge.

18. An end effector according to claim 17, wherein the chamfered edge and/or radius edge connects an outer surface of the cutting blade to an inner surface of the cutting blade.

19. An end effector according to claim 18, wherein the chamfered edge and/or radius edge is angled such that the inner surface of the cutting blade extends further than the outer surface of the cutting blade.

20. An end effector according to claim 18, wherein the one or more tissue engaging cutting points are located at the inner surface of the cutting blade.

21. An end effector according to claim 17, wherein the chamfered edge and/or radius edge is angled at an angle between 10 and 60 degrees with respect to an axial plane to a longitudinal plane of the shaver arrangement.

22. An end effector according to claim 12, wherein:
the shaver arrangement is a rotary shaver arrangement;
the outer member is an outer tubular member with a cutting window at the distal end thereof;
the cutting window has at least one sharpened edge to form the cutting blade; and
the tissue engaging cutting points are located radially inwards from the outer tubular member such that there are no tissue engaging cutting points capable in use of snagging and/or cutting tissue located on or outwards of an outer circumference of the outer tubular member.

23. An end effector according to claim 22, wherein the outer tubular member has a central passageway and the cutting window is a first cutting window, and the arrangement further comprises an inner tubular member rotatably mounted in the central passageway of the outer tubular member and providing a central suction lumen, the inner tubular member having a second cutting window at the distal end thereof such that the first and second cutting windows align when the inner tubular member is rotated to a first position.

24. An electrosurgical instrument comprising:
an end effector according to claim 11; and
an operative shaft having RF electrical connections operably connected to the active electrode, and drive componentry operably connected to the shaver arrangement to drive the shaver arrangement to operate in use.

25. An electrosurgical system, comprising:
an RF electrosurgical generator; and
an electrosurgical instrument according to claim 24, the arrangement being such that in use the RF electrosurgical generator supplies an RF coagulation or ablation signal via the RF electrical connections to the active electrode.
